(19) United States
(12) Patent Application Publication
(10) Pub. No.: US 2013/0158637 A1
(43) Pub. Date: Jun. 20, 2013

(54) Ice Bag Closures and Methods for Manufacturing Ice Bag Closures

(71) Applicant: Shahid Sheikh, Venice, CA (US)

(72) Inventor: Shahid Sheikh, Venice, CA (US)

(21) Appl. No.: 13/327,636

(22) Filed: Dec. 15, 2011

(51) Int. Cl. A61F 7/10; B31B 1/90; B65D 33/24; B31B 29/84

(52) U.S. Cl. 607/114; 493/213; 493/214; 383/80

(57) ABSTRACT

An ice bag is described having a bladder formed with an opening, a threaded receiver coupled to the bladder at the opening, a threaded cap having a first portion formed of a rigid material and a second portion formed of an overmold material, the second portion providing a seal between the receiver and cap with the cap threaded into the receiver.

### FIELD

The present invention pertains generally to ice bags. More particularly, the present invention pertains to ice bag closures and methods for manufacturing ice bag closures.

### BACKGROUND

Ice bags, which are also commonly referred to as medical ice bags or English style ice bags, generally include a bladder that is made of a waterproof material, such as an impervious cloth-like fabric, and is shaped having a relatively wide mouth or opening allowing ice cubes to be passed through the opening and into the bladder. A closure assembly is typically provided to open, close and seal the bladder opening. For example, the closure assembly may include a receiver that is fastened or clamped to the bladder material and a screw cap that threads into the receiver.

To use the ice bag, the screw cap may be removed to fill the bladder with ice, ice water, chilled or heated water or other thermal mixtures or solutions at a selected temperature. Once filled, the cap is replaced and the ice bag can be applied to a portion of the user's body. For example, the ice bag may be manually applied and held against a portion of the user's body or a wrap may be used to press and secure the ice bag against a portion of the user's body. In this regard, U.S. Pat. No. 6,589,272 granted on Jul. 8, 2003 to Sheikh and titled “Thermal Pack Retaining Apparatus” discloses a joint-specific apparatus that reliably retains a cold pack in a preselected position and under compression adjacent to an anatomical structure such as a limb or joint, the entire contents of U.S. Pat. No. 6,589,272 are hereby incorporated by reference herein.

Ice bags can be used to reduce pain, swelling and inflammation, for example, during rehabilitation of injured joints and muscles and in post-surgical patients. For example, joints may be injured during exercise, while engaging in sporting or on-the-job activities or during an accident such as a fall. These injuries can include circumstances in which a muscle, ligament or tendon is sprained, torn or otherwise traumatized.

In the past, a removable gasket has been used to create a water-tight seal between the screw cap and receiver of an ice bag. However, the removable gasket is often lost or damaged rendering the ice bag unusable. In addition, the small gasket, if removed from the cap, can present a choke hazard for small children and infants. Moreover, debris which enters the gap between the gasket and cap can cause the closure assembly to leak.

In light of the above, Applicant discloses Ice Bag Closures and Methods for Manufacturing Ice Bag Closures.

### SUMMARY

In one aspect of the present disclosure, an ice bag is described herein which comprises; a bladder formed with an opening; a threaded receiver coupled to the bladder at the opening; a threaded cap having a first portion formed of a rigid material and a second portion formed of an overmold material, the second portion providing a seal between the receiver and cap with the cap threaded into the receiver.

In one embodiment of this aspect, the receiver has a first portion formed of a rigid material and a second portion formed of an overmold material, the second portion providing a seal between the receiver and bladder with the bladder coupled to the receiver.

In one arrangement of this aspect, the first portion of the cap comprises a hollow cylindrical portion and a flange and wherein the overmold material overlays a surface of the flange.

In one setup of this aspect, the flange surface is formed with a recess to increase bond strength between the overmold material and the flange.

In one implementation of this aspect, the flange extends to lip formed with a plurality of cutouts and the overmold material overlays a surface of the lip and establishes a plurality of grips for the cap within the cutouts.

In a particular embodiment of this aspect, the overmold material is a thermoplastic elastomer.

In one arrangement of this aspect, the rigid material is a plastic.

In another aspect, an ice bag is described herein which comprises; a first threaded component and a second component, the first component having a first portion formed of a rigid material and a second portion formed of an elastomer fixedly attached to the first portion to provide a seal between the first and second components when the first and second components are coupled together.

In one embodiment of this aspect, the first threaded component is a receiver and the second component is a bladder.

In one embodiment of this aspect, the first threaded component is a receiver and the second component is a cap.

In one implementation of this aspect, the first threaded component is a cap and the second component is a receiver.

In a particular implementation of this aspect, the elastomer is fixedly attached to the first portion using an adhesive.

In a particular embodiment of this aspect, the elastomer is an overmold material.

In one arrangement of this aspect, the second component has a first portion formed of a rigid material and a second portion formed of an elastomer fixedly attached to the first portion of the second component to provide a seal between the second component and a third component when the second and third components are coupled together.

In another aspect, a method of producing an ice bag is described herein comprising the steps or acts of; providing a bladder formed with an opening; molding a first threaded component formed of a rigid material and a second component formed of a rigid material; and overlying a thermoplastic elastomer material on a portion of the first threaded component in an overmolding process to produce a seal.

In one embodiment of this aspect, the overmolding process is a multi-shot injection molding process.

In one implementation of this aspect, the overmolding process is an insert molding processes.

In one implementation of this aspect, the first threaded component is a cap.

In a particular implementation of this aspect, the first threaded component is a receiver.

In a particular embodiment of this aspect, the step of overlying a thermoplastic elastomer material overlays a thermoplastic elastomer material on a receiver flange to produce a seal between the receiver and a cap.

In another aspect of the present disclosure, an ice bag is described herein which comprises; a bladder formed with an opening; a threaded receiver coupled to the bladder at the opening, the receiver having a first portion formed of a rigid material and a second portion formed of an overmold material, the second portion providing a seal between the receiver and bladder with the bladder coupled to the receiver; and a threaded cap.

In another aspect, an ice bag is described herein made by the process comprising the steps or acts of; providing a bladder formed with an opening;

molding a first threaded component formed of a rigid material and a second component formed of a rigid material; and overlying a thermoplastic elastomer material on a portion of the first threaded component in an overmolding process to produce a seal.

### BRIEF DESCRIPTION OF THE DRAWINGS

FIG. 1 shows a perspective view of an ice bag;

FIG. 2 shows a perspective view of a receiver for use in the ice bag shown in FIG. 1;

FIG. 3 shows a sectional view of the receiver shown in FIG. 2 as seen along line 3-3 in FIG. 2;

FIG. 4 shows a perspective view of an embodiment of a cap for use in the ice bag shown in FIG. 1;

FIG. 5 shows another perspective view of the cap shown in FIG. 4;

FIG. 6 shows a sectional view of the cap of FIG. 4 as seen along line 6-6 in FIG. 4;

FIG. 7 shows a perspective view of another embodiment of a cap for use in the ice bag shown in FIG. 1;

FIG. 8 shows another perspective view of the cap shown in FIG. 7;

FIG. 9A shows a sectional view of the cap of FIG. 7 as seen along line 9A-9A in FIG. 7 showing a section of the cap in which the lip fully extends to a lip edge;

FIG. 9B shows a sectional view of the cap of FIG. 7 as seen along line 9B-9B in FIG. 7 showing a section of the cap in which a cutout is formed in the lip to establish a grip for the cap;

FIG. 10 shows a perspective view of another embodiment of a cap for use in the ice bag shown in FIG. 1;

FIG. 11 shows a sectional view of the cap of FIG. 10 as seen along line 11-11 in FIG. 10;

FIG. 12 shows a perspective view of another embodiment of a cap for use in the ice bag shown in FIG. 1;

FIG. 13 shows a sectional view of the cap of FIG. 12 as seen along line 13-13 in FIG. 12; and

FIG. 14 shows an exploded, perspective view illustrating the assembly of another embodiment of a cap for use in the ice bag shown in FIG. 1.

### DETAILED DESCRIPTION

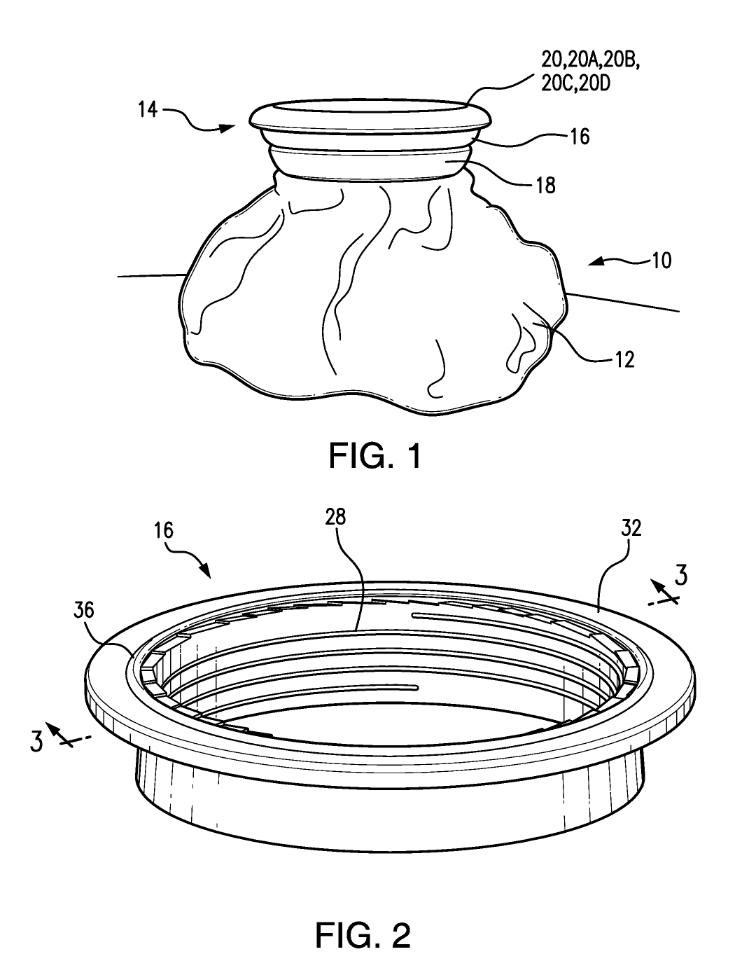

Referring to FIG. 1, an ice bag for holding a material contents at a selected temperature and applying the material contents to a portion of a person's anatomy to facilitate a thermal exchange between the material contents and the portion of a person's anatomy is shown and generally designated 10.

As shown in FIG. 1, the ice bag 10 includes a bladder 12 that is shaped to envelop a volume suitable for holding a material contents such as ice. The bladder 12 is generally formed of a water impervious material such as an elastomeric material or a cloth that has been coated with a water impervious coating. As shown, the bladder forms an opening allowing fluids and solids such as ice to be delivered into and removed from the bladder 12. The bladder may be preformed, e.g. heat molded, into the desired shape or, as shown, may be formed of a sheet, the edges of which are gathered together and pleated to form the opening.

As detailed further below, FIG. 1 shows that the ice bag 10 includes a closure 14 at the opening of the bladder 12 to close and prevent the contents in the bladder 12 from leaking. As shown, the closure 14 includes a receiver 16 that is affixed to the bladder opening, e.g. pleats, using a clamp ring 18 that can sandwich the pleated fabric between the clamp ring 18 and receiver 16, for example, using a swaging or clamping technique. Alternative techniques for attaching the bladder to the receiver may be used, for example, the receiver may be bonded to the bladder. It can be further seen in FIG. 1 that the closure 14 includes a removable cap 20 that is threaded into the receiver 16.

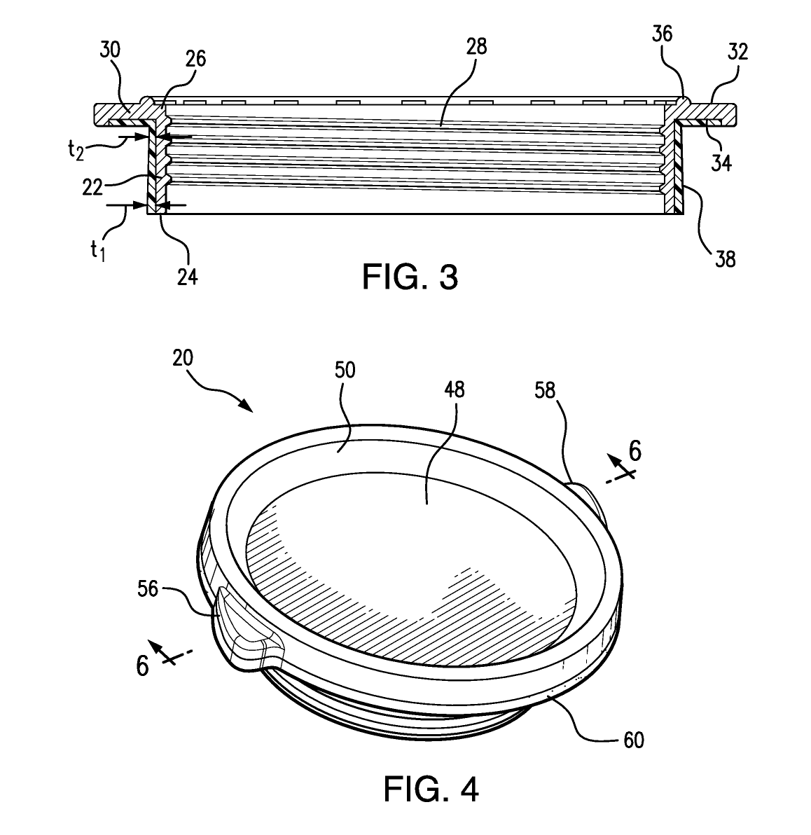

FIGS. 2 and 3 show an embodiment of a receiver 16 for use in the ice bag 10 shown in FIG. 1. As seen there, the receiver 16 can include a hollow cylindrical portion 22 that extends from a first end 24 to a second end 26. One or more thread features 28 extend from the inner wall of the cylindrical portion 22 to establish a set of internal, female threads for the receiver 16. For example, the cylindrical portion may have an internal cylinder diameter of about 1.5 to 3 inches (38-76 mm). Receiver 16 includes an annular flange 30 that extends outwardly from cylinder portion 22 at second end 26 and establishes a first flange surface 32 and opposed second flange surface 34. Raised protrusion 36 having a rounded profile is formed on surface 32 and extends, unbroken, annularly around the entire length of the first flange surface 32. Cylindrical portion 22, thread features 28, annular flange 30 and raised protrusion 36 can be made of a one-piece rigid material, for example, a molded rigid plastic.

The term “rigid material” as used herein is a broad term, and is to be given its ordinary and customary meaning to a person of ordinary skill in the art (and is not to be limited to a special or customized meaning), and refers without limitation to a material that does not substantially compress, deform or change shape during an ordinary use, and, if applicable, for a plurality of use cycles. Examples of rigid materials include, but are not necessarily limited to metals, wood and plastics including Acetal, Acrylonitrile Butadiene Styrene, Polycarbonate, Polypropylene, Polyethylene, Polystyrene, High Impact Polystyrene, Polymethylmethacrylate, Polyesters, Copolyester, Polyamide, Polybutylene Terephthalate, Polyphenylene Oxide, Glycol Modified Polyethylene Terephthalate and their blends and alloys.

FIGS. 2 and 3 further show that the receiver 16 may include an overmold material overlying the outer surface of cylindrical portion 22 and a portion of the surface 34 of flange 30. As shown, the overmold material may be thicker, “t₁” near the first end 24 of the cylindrical portion 22 than near the second end 26, “t₂” of the cylindrical portion 22 (i.e. “t₁”>“t₂”) to prevent the clamp ring 18 (see FIG. 1) from backing off of the receiver 16 (in use). Typically, the overmold material is a thermoplastic elastomer that is molded onto the rigid plastic cylindrical portion 22 and flange 30 using an overmolding process. For example, the overmold material may have a thickness, “t₁” in the range of about 0.3 to 10 mm and a thickness, “t₂” in the range of about 0.1 to 5 mm.

During assembly of the ice bag 10 shown in FIG. 1, the first end 24 of the cylindrical portion 22 is inserted into the opening formed in the bladder 12 until the bladder abuts the overmold material overlying the surface 34 of the flange 30. The clamp ring 18 is then installed on the assemble to sandwich and compress the bladder, e.g. pleats and overmold material between the clamp ring 18 and the rigid material portion of the receiver 16, providing a water-tight seal between the receiver 16 and bladder 12.

The term “overmolding” as used herein is a broad term, and is to be given its ordinary and customary meaning to a person of ordinary skill in the art (and is not to be limited to a special or customized meaning), and refers without limitation to a molding process in which an overmold material is molded onto a substrate material and becomes bonded to the substrate material without the use of a third material such as an adhesive. For example, an overmold material consisting of a thermoplastic elastomer (TPE) can be molded onto a rigid substrate. Types of overmolding processes can include, but are not necessarily limited to, multi-shot injection molding processes and insert molding processes. In a multi-shot injection molding process, two (or more) materials, such as a rigid substrate material and overmold material are shot into the same mold during the same molding cycle. In an example of an insert molding process, a pre-molding insert such as a rigid substrate can be placed into a mold and the overmold material can be introduced into the mold where it contacts and adheres to the pre-molded insert.

The term “overmold material” as used herein is a broad term, and is to be given its ordinary and customary meaning to a person of ordinary skill in the art (and is not to be limited to a special or customized meaning), and refers without limitation to a material that is bonded to a substrate material by an overmolding process. Overmold materials include thermoplastic elastomers and other materials that have been bonded to a substrate material by an overmolding process. In some cases, the overmold material may be softer than the substrate material. For example, the overmold material may have a durometer hardness between about 10 shore OO and 80 shore A.

The term “thermoplastic elastomer” as used herein is a broad term, and is to be given its ordinary and customary meaning to a person of ordinary skill in the art (and is not to be limited to a special or customized meaning), and refers without limitation to an elastomeric material that can be applied to a substrate in an overmolding process. Examples of thermoplastic elastomers include, but are not necessarily limited to Copolyamides, Copolyesters, thermoplastic Urethanes, thermoplastic Vulcanizates, Olefinic Copolymers and Styrenic block copolymers. Thermoplastic elastomers having proprietary compositions are often sold under tradenames such as Dynaflex®, Versaflex®, Versollon™, Versalloy® and Noryl®.

The term “elastomer” as used herein is a broad term, and is to be given its ordinary and customary meaning to a person of ordinary skill in the art (and is not to be limited to a special or customized meaning), and refers without limitation to a material that can be deformed, for example, compressed, during ordinary use and subsequently recovers to substantially its original shape when stresses are removed, for a plurality of stress-recovery cycles. Examples of elastomers include, but are not necessarily limited to natural and synthetic rubber including silicone and the thermoplastic elastomers listed above.

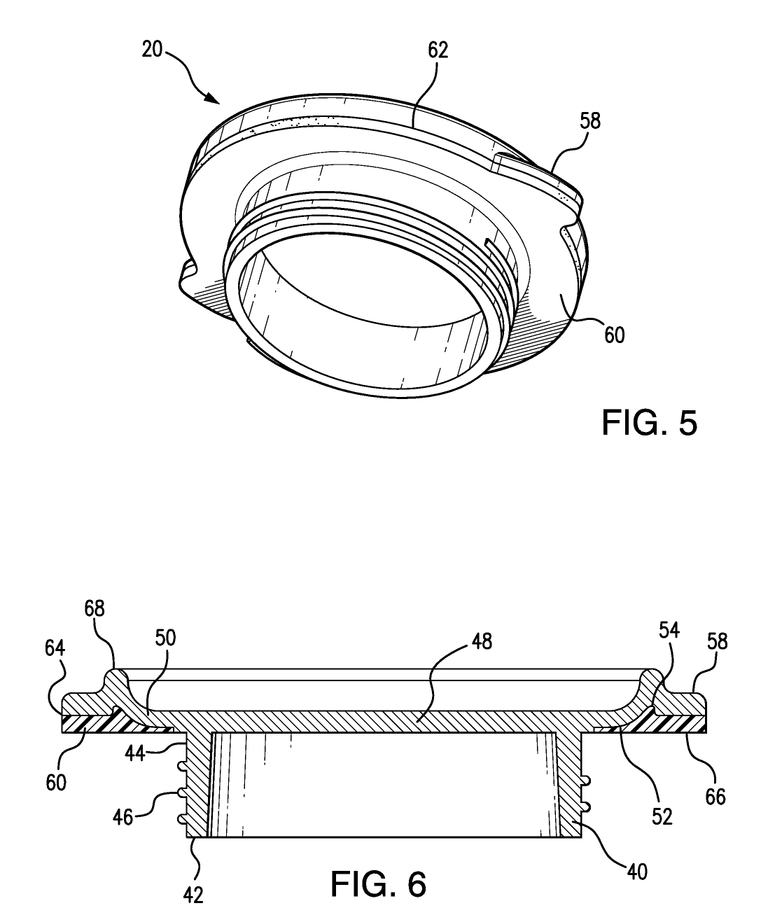

FIGS. 4-6 show an embodiment of a cap 20 for use in the ice bag 10 shown in FIG. 1. As shown, the cap 20 includes a hollow cylindrical portion 40 that extends from a first end 42 to a second end 44. One or more thread features 46 extend from the outer wall of the cylinder portion 40 to establish a set of external, male threads for the cap 20. Cap 20 includes a generally flat cover 48 closing the hollow cylindrical portion 40 at the second end 44. An annular flange 50 that extends outwardly from the cover 48 at second end 44 and establishes a flange surface 52. FIGS. 4 and 6 show that the flange 50 is non-flat and extends away from the cover 48 and out of the plane of the cover 48. FIG. 6 further shows that an annular recess 54 is formed in the flange surface 52 that extends around the entire length of the flange 50. A pair of grips 56, 58 extend from flange 50 to facilitate screwing the cap 20 into the receiver 16 (shown in FIG. 2). Cylindrical portion 40, thread features 46, annular flange 50 and cover 48 can be made of a one-piece rigid material, for example, a molded rigid plastic.

FIGS. 4-6 further show that the cap 20 includes an overmold material 60 overlying the flange surface 52 and extending from the cylindrical portion, into recess 54, and beyond recess 54 to the edge 62 of the flange 50 (and the edge 64 of the grips 56, 58). As shown, the surface 66 of the overmold opposite the flange 50 is substantially flat. Typically, the overmold material is a thermoplastic elastomer that is molded onto the rigid plastic flange 50 and grips 56,58 using an overmolding process. For example, the overmold material may have a thickness at the edge 62 of the flange 50 in the range of about 0.5 to 15 mm. Recess 54 is provided to increase the bond strength between overmold material 60 and flange 50 (relative to a flat flange) and increase moldability of the rigid material due by providing a flange having a more uniform thickness from the cylindrical portion 40 to the flanged end 68.

The installation of the cap 20 into the receiver 16 can best be appreciated by cross referencing FIGS. 6 and 3. To install, the first end 42 of the cap 20 is inserted in the second end 26 of the receiver 16 to engage thread portions 46 with thread portions 28. The cap is then rotated relative to the receiver (using grips 56, 58) until the overmold 60 contacts the raised protrusion 36. Additional rotation of the cap 20 is then applied until the raised protrusion 36 compresses the overmold material 60 between the raised protrusion 36 and flange 50 to establish a water tight seal between the receiver 16 and cap 20.

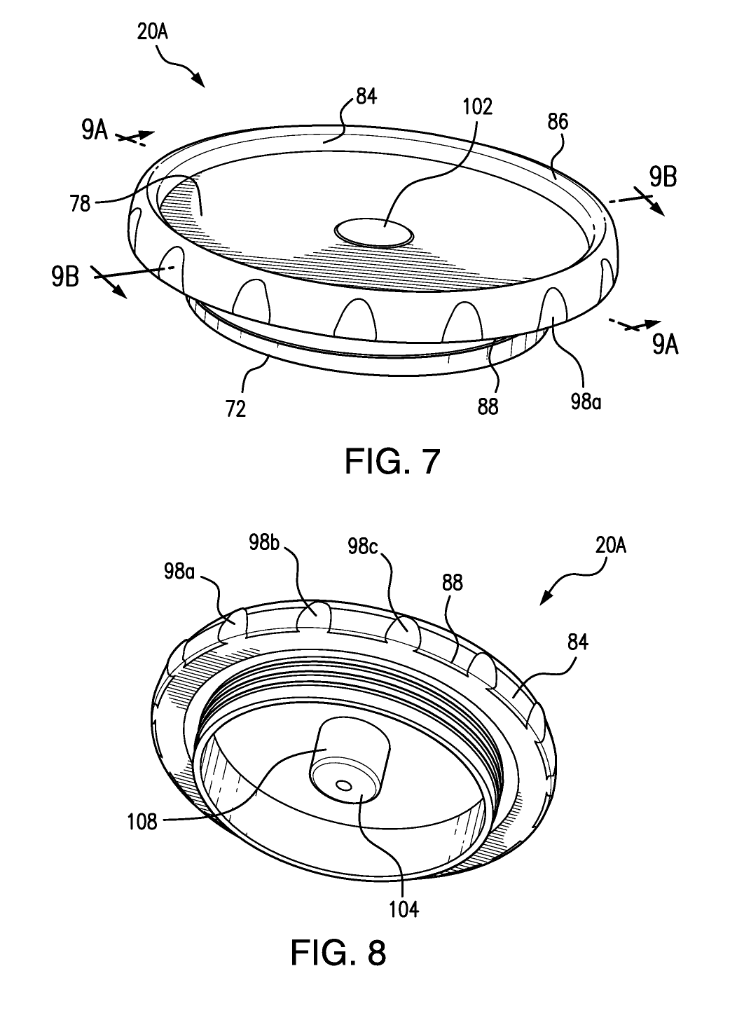

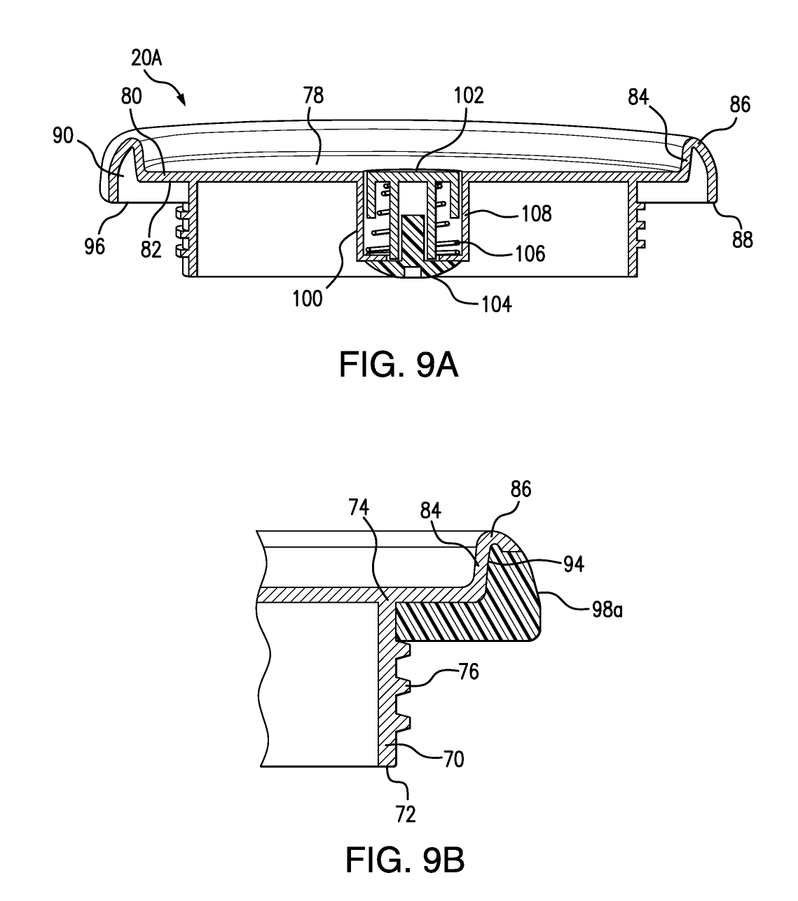

FIGS. 7-9A and 9B show another embodiment of a cap 20A for use in the ice bag 10 shown in FIG. 1. As shown, the cap 20A includes a hollow cylindrical portion 70 that extends from a first end 72 to a second end 74. One or more thread features 76 extend from the outer wall of the cylinder portion 70 to establish a set of external, male threads for the cap 20A. Cap 20A includes a generally flat cover 78 closing the hollow cylindrical portion 70 at the second end 74. An annular flange 80 that extends outwardly from the cover 78 at second end 74 of cylindrical portion 70 and establishes a flange surface 82. FIGS. 9A and 9B show that the flange 80 is substantially flat and extends away from the cover 78 to a lip 84. As shown, lip 84 curls from the flange 80 through an apex 86 to an edge 88. From the apex 86 to the edge 88, the lip is formed with a plurality of cutouts that are spaced around the lip 84. In some embodiments, the cutouts may be equally spaced around the lip 84. Cylindrical portion 70, thread features 76, annular flange 80, lip 84 and cover 78 can be made of a one-piece rigid material, for example, a molded rigid plastic.

FIGS. 7-9 further show that the cap 20A includes an overmold material 90 overlying the flat flange surface 82 and inner surface 94 of curled lip 84. As shown, the surface 96 of the overmold material 90 opposite the flange 80 is substantially flat. Typically, the overmold material is a thermoplastic elastomer that is molded onto the rigid plastic flange 80 and lip 84 using an overmolding process. For example, the overmold material may have a thickness on the flange 80 in the range of about 0.5 to 25 mm. FIGS. 7, 8 and 9A show that the overmold material 90 extends to the edge 88 of the lip 84 to create a plurality of grips (of which grips 98a-c are labeled). The overmold material can be formed to create grips 98a-c that are flush with or slightly raised above the surface of the lip 84.

FIGS. 7-9 also illustrate that a valve 100 may be incorporated into the cap 20A (or any of the other caps 20, 20B, 20C or 20D described herein). Alternatively, cap 20A may be configured without valve 100 in which case the cover 78 would be continuous like the cover 48 shown in FIG. 4. As shown in FIGS. 7-9A, the valve 100 can include a button 102 that is manually depressible to release air from the bladder 12 (shown in FIG. 1) when the bladder is partially collapsed. As best seen in FIG. 9A, the valve 100 includes a valve-stem 104 that is biased in a closed position (shown closed in FIG. 9A) by spring 106 to cover an opening in chamber 108. Depressing button 102 moves stem 104 to allow air from the bladder to escape through the valve 100.

The installation of the cap 20A into the receiver 16 can best be appreciated by cross-referencing FIGS. 3 and 9A. To install, the first end 72 of the cap 20A is inserted in the second end 26 of the receiver 16 to engage thread portions 76 with thread portions 28. The cap is then rotated relative to the receiver (using grips 98a-c) until the overmold material 90 contacts the raised protrusion 36. Additional rotation of the cap 20A can then applied until the raised protrusion 36 compresses the overmold material 90 between the raised protrusion 36 and flange 80 to establish a watertight seal between the receiver 16 and cap 20A. Once sealed, the button 102 can be depressed and the bladder 12 collapsed to devoid the bladder 12 of air. Removing air from the bladder 12 can improve the ability of the user to conform the ice bag 10 to the targeted anatomical region.

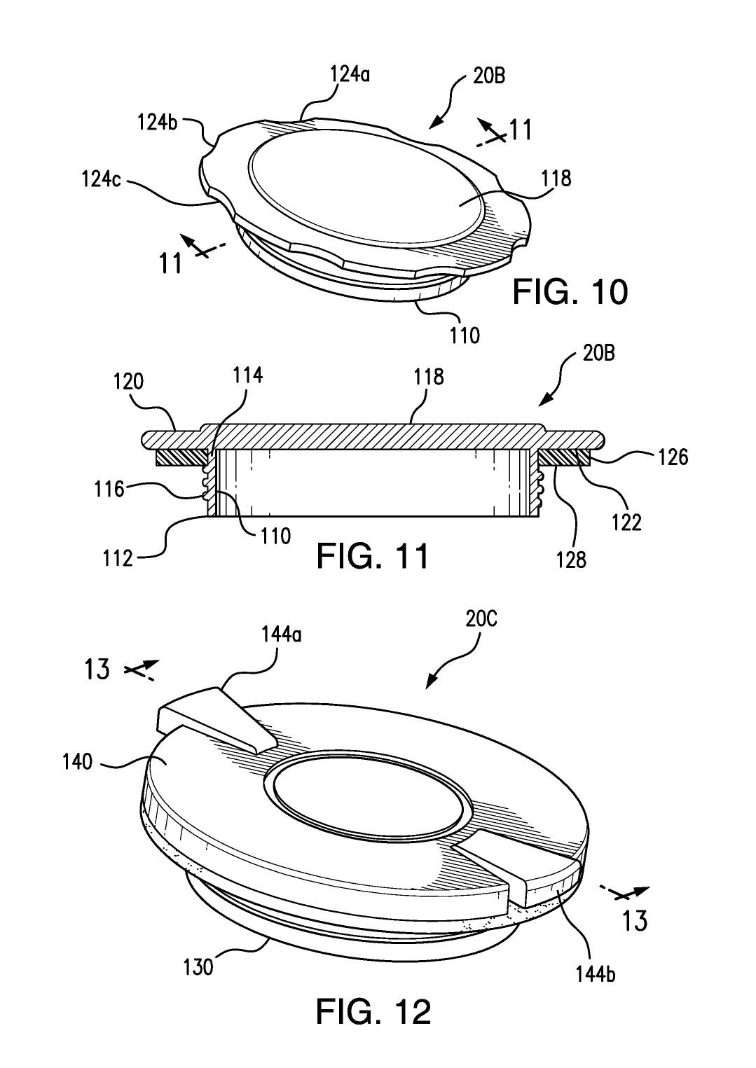

FIGS. 10 and 11 show another embodiment of a cap 20B for use in the ice bag 10 shown in FIG. 1. As shown, the cap 20B includes a hollow cylindrical portion 110 that extends from a first end 112 to a second end 114. One or more thread features 116 extend from the outer wall of the cylinder portion 110 to establish a set of external, male threads for the cap 20B. Cap 20B includes a generally flat cover 118 closing the hollow cylindrical portion 110 at the second end 114. An annular flange 120 that extends outwardly from the cover 118 at second end 114 of cylindrical portion 110 and establishes a substantially flat flange surface 122. As shown, flange 120 extends to a flange gear-shaped flange edge having alternating concave-convex edge contours to provide convex grip surfaces of which convex grip surface 124a-c have been labeled. Cylindrical portion 110, thread features 116, annular flange 120 and cover 118 can be made of a one-piece rigid material, for example, a molded rigid plastic.

FIGS. 10 and 11 further show that the cap 20B includes an overmold material 126 overlying the flat flange surface 122. For example, the overmold material may have a thickness on the flange surface 122 in the range of about 0.5 to 15 mm. As shown, the surface 128 of the overmold material 126 opposite the flange 120 is substantially flat. Typically, the overmold material is a thermoplastic elastomer that is molded onto the rigid plastic flange 120 using an overmolding process.

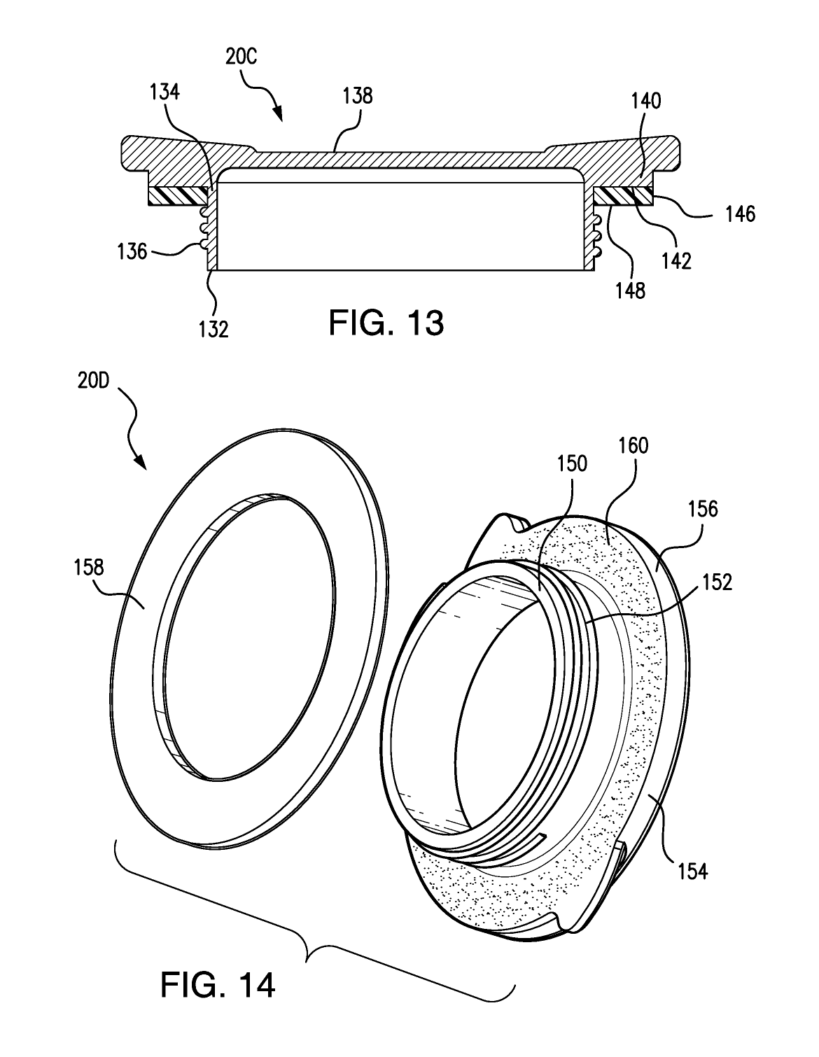

FIGS. 12 and 13 show another embodiment of a cap 20C for use in the ice bag 10 shown in FIG. 1. As shown, the cap 20C includes a hollow cylindrical portion 130 that extends from a first end 132 to a second end 134. One or more thread features 136 extend from the outer wall of the cylinder portion 130 to establish a set of external, male threads for the cap 20C. Cap 20C includes a generally flat cover 138 closing the hollow cylindrical portion 130 at the second end 134. An annular flange 140 that extends outwardly from the cover 138 at second end 134 of cylindrical portion 130 and establishes a substantially flat flange surface 142. As shown, grips 144a,b extend from flange 140.

Cylindrical portion 130, thread features 136, annular flange 140, cover 138 and grips 144a,b can be made of a one-piece rigid material, for example, a molded rigid plastic.

FIGS. 12 and 13 further show that the cap 20C includes an overmold material 146 overlying the flat flange surface 142. As shown, the surface 148 of the overmold material 146 opposite the flange 140 is substantially flat. Typically, the overmold material is a thermoplastic elastomer that is molded onto the rigid plastic flange 140 using an overmolding process. For example, the overmold material may have a uniform thickness on the flange surface 142 in the range of about 0.5 to 15 mm.

FIG. 14 shows another embodiment of a cap 20D for use in the ice bag 10 shown in FIG. 1. As shown, the cap 20D includes a hollow cylindrical portion 150 One or more thread features 152 extend from the outer wall of the cylinder portion 150 to establish a set of external, male threads for the cap 20D. Cap 20D includes a generally flat cover closing the hollow cylindrical portion 150. An annular flange 154 extends outwardly from the cover and establishes a substantially flat flange surface 156. Cylindrical portion 150, thread features 152, annular flange 154 and cover can be made of a one-piece rigid material, for example, a molded rigid plastic. Alternatively, or in addition to including an overmold material overlying a flange surface of cap 20, 20A, 20B or 20C, an overmold material overlying a flange surface 32 of receiver 16 can be employed to provide a seal between the receiver 16 and cap 20, 20A, 20B or 20C.

FIG. 14 further illustrates that a ring shaped gasket 158 can be bonded to the flat flange surface 156 using an adhesive 160. Once bonded, the gasket 158 is affixed in place on the flange 154. Typically, the gasket 158 is made of an elastomer. Alternatively, or in addition to adhesively bonding a gasket to flange surface 156 of cap 20D, a gasket may be adhesively bonded to flange surface 32 of receiver 16 to provide a seal between the receiver 16 and cap 20D.

Cap 20, 20A, 20B, 20C and 20D may be used with the receiver 16 shown or, alternatively, with a receiver that does not include an overmold material for coupling the receiver to a bladder (receiver not shown).

While the particular embodiment(s) are described and illustrated in this patent application in the detail required to satisfy 35 U.S.C. 112, it is to be understood by those skilled in the art that the above-described embodiment(s) are merely examples of the subject matter which is broadly contemplated by the present application. Reference to an element in the following Claims in the singular, is not intended to mean, nor shall it mean in interpreting such Claim element “one and only ^{one} unless explicitly so stated, but rather “one or more”. All structural and functional equivalents to any of the elements of the above-described embodiment(s) that are known, or later come to be known to those of ordinary skill in the art, are expressly incorporated herein by reference and are intended to be encompassed by the present Claims. It is not intended or necessary for a device or method discussed in the Specification as an embodiment, to address or solve each and every problem discussed in this Application, for it to be encompassed by the present Claims. No element, component, or method step in the present disclosure is intended to be dedicated to the public regardless of whether the element, component, or method step is explicitly recited in the Claims. No claim element in the appended Claims is to be construed under the provisions of 35 U.S.C. 112, sixth paragraph, unless the element is expressly recited using the phrase “means for” or, in the case of a method claim, the element is recited as a “step” instead of an “act”.

What is claimed is:

1. An ice bag comprising;
a bladder formed with an opening;
a threaded receiver coupled to said bladder at said opening;
a threaded cap having a first portion formed of a rigid material and a second portion formed of an overmold material, said second portion providing a seal between said receiver and cap with said cap threaded into said receiver.

2. An ice bag as recited in claim 1 wherein said receiver has a first portion formed of a rigid material and a second portion formed of an overmold material, said second portion providing a seal between said receiver and bladder with said bladder coupled to said receiver.

3. An ice bag as recited in claim 1 wherein said first portion of said cap comprises a hollow cylindrical portion and a flange and wherein said overmold material overlays a surface of said flange.

4. An ice bag as recited in claim 3 wherein said flange surface is formed with a recess to increase bond strength between said overmold material and said flange.

5. An ice bag as recited in claim 3 wherein said flange extends to lip formed with a plurality of cutouts and said overmold material overlays a surface of said lip and establishes a plurality of grips for said cap within said cutouts.

6. An ice bag as recited in claim 1 wherein said overmold material is a thermoplastic elastomer.

7. An ice bag as recited in claim 1 wherein said rigid material is a plastic.

8. An ice bag comprising;
a first threaded component and a second component, said first component having a first portion formed of a rigid material and a second portion formed of an elastomer fixedly attached to said first portion to provide a seal between said first and second components when said first and second components are coupled together.

9. An ice bag as recited in claim 8 wherein said first threaded component is a receiver and said second component is a bladder.

10. An ice bag as recited in claim 8 wherein said first threaded component is a receiver and said second component is a cap.

11. An ice bag as recited in claim 8 wherein said first threaded component is a cap and said second component is a receiver.

12. An ice bag as recited in claim 8 wherein said elastomer is fixedly attached to said first portion using an adhesive.

13. An ice bag as recited in claim 8 wherein said elastomer is an overmold material.

14. An ice bag as recited in claim 8 wherein said second component has a first portion formed of a rigid material and a second portion formed of a elastomer fixedly attached to said first portion of said second component to provide a seal between said second component and a third component when said second and third components are coupled together.

15. A method for producing an ice bag comprising the steps of;
providing a bladder formed with an opening;
molding a first threaded component formed of a rigid material and a second component formed of a rigid material; and
overlying a thermoplastic elastomer material on a portion of said first threaded component in an overmolding process to produce a seal.

16. The method as recited in claim 15 wherein said overmolding process is a multi-shot injection molding process.

17. The method as recited in claim 15 wherein said overmolding process is an insert molding processes.

18. The method as recited in claim 15 wherein said first threaded component is a cap.

19. The method as recited in claim 15 wherein said first threaded component is a receiver.

20. The method as recited in claim 19 wherein said step of overlying a thermoplastic elastomer material overlays a thermoplastic elastomer material on a receiver flange to produce a seal between said receiver and a cap.
